(19) United States
(12) Patent Application Publication
(10) Pub. No.: US 2004/0236373 A1
(43) Pub. Date: Nov. 25, 2004

(54) Surgical method for suturing tendons/ligaments to bones

(76) Inventor: William E. Anspach III, Stuart, FL (US)

Correspondence Address: Norman Friedland, Suite 400, 11300 US Highway One, North Palm Beach, FL, 33408, US

(21) Appl. No.: 10/441,835

(22) Filed: May 20, 2003

(51) Int. Cl. A61B 17/04

(52) U.S. Cl. 606/232

(57) ABSTRACT

The repair of torn tendon/ligament by anchoring the tendon to a anchor affixed to the bone is by a suture judiciously looped in the tissue to form a double strand holding the tending to cinch to the bone trough and securing the strand to and downstream of the anchor. This enhances the pull-out resistance of the suture from the tendon. The suture, secured by a knot or clamp, is at the end of the tendon and not under it so as to allow the surgeon to maintain constant apposition to the bone while the knot is being tied. The knot is in the bone trough and not over the tendon avoiding abrasion of the suture by other parts of the body. When the operation is done by arthroscopic surgery the ability for cinching the tissue down to the bone and assessing how tightly the tissue is apposed to the bone and tying the knot while maintaining secure apposition of the tendon to the bone is improved.

### CROSS REFERENCES

This invention relates to the invention co-pending patent application entitled SUTURE CLAMP filed by WILLIAM E. ANSPACH III, filed contemporaneously and is commonly assigned to The Anspach Effort, Inc. and identified by Attorney Docket No. N1048 and is incorporated herein in its entirety by reference.

### TECHNICAL FIELD

This invention relates to the surgical procedure for attaching tendons/ligaments (tissues) to bones of a patient and particularly, to the method for repair of torn tissue by either arthroscopic or general surgery where the tissue is attached to the bone by uniquely tying the suture to the tendon and then to the anchor where the knot or clamp is located in the trough of the bone.

### BACKGROUND OF THE INVENTION

There are a sundry of tissue fixation systems that are utilized by surgeons in performing surgery for repair of tissue that requires the securing of the tissue of a patient to the bone. Many or most of these procedures include a pin, screw or suture anchor that is affixed to the bone and looping the suture over or through the tendon and finally, securing the suture to the anchor with a well known knot or clamping device. Other surgical procedure fix torn tendons without the use of sutures by impaling the tissue with the pin, screw, or anchor and into the bone or a hole drilled in the bone. This invention is principally concerned with the method of inserting the suture in the tendon or ligament and tying the suture to the anchor affixed to the bone. Although, as it will become clear from the description that follows, the method can also be applied where the tissue is not secured to the bone.

As is well known to those skilled in this technology, in repairing torn tissue in a rotator cuff surgical procedure, for example, after the incision, the surgeon will contour the bone surface along the target area adjacent the tendon where the repair is being performed typically with an appropriate burring instrument so as to remove the hard surface of the bone. The surgeon will attempt to cinch the tissue down to the bone where the bone is roughened and/or scored and where there is a propensity in the bone structure to grow and adhere to the tissue. When the procedure is done by arthroscopic surgical procedure, it is apparent that the surgeon would have a difficult time in tying the knot to or clamping the suture because of the size of the opening in the body of the patient. Not only is it extremely difficult for the surgeon to assess how tightly the tissue is apposed to the bone, it is especially difficult for the surgeon to tie the knot while maintaining secure apposition of the tendon to the bone. While sliding knots have been devised to attempt to cure this problem, sliding knots are cumbersome and difficult to tie.

In heretofore procedures where fixation systems use sutures and tie the suture with a knot or clamp, the location of the knot is such that it tends to be in proximity to a nearby bone or other member of the patient where it can rub and hence, prematurely rupture. This, of course, would require reopening the incision and retying the tissue. Additionally, it is common practice for the surgeon to roughen the bone in order to attain the bonding process of the tendon/ligament and bone. This procedure is not known to be without pain and obviously, a recurrence of the tying procedure subjects the patient to additional pain. It is, of course, abundantly important that the suture does not break requiring a re-visit to the target area.

Perhaps the best way to illustrate the problem that is solved by this invention is to consider the details of FIG. 1 which is a schematic illustration of tying of the tendon with a suture and to a suture anchor. In this method the bone is roughened to define a trough and drilled to accommodate the anchor 10. In this illustration the surgical rivet 11 is driven into the bone 12. After the bone has been prepared and the anchor is mounted into the bone, the suture 14 is looped around a portion of or through the tendon/ligament 16 and then into a slot or eyelet 18 formed on the end of the rivet anchor 10. The suture 14 is then tied together by the surgeon with a suitable knot. As is apparent from FIG. 1, by tying the tissue, as is shown in this illustration, the knot is located above the tendon and in the event it is in close proximity to another appurtenance, such as bone, the motion on the patient has the propensity to cause rubbing of the suture and hence, rupturing or breaking thereof

Essentially, this invention addresses at least three problems that the present invention solves. Namely, the invention enhances the pull-out resistance that typically is the most frequent cause of failure of tendon repair; obviates the problem of cinching the tissue down to the bone and tying the knot while maintaining secure apposition of the tendon to the bone; and locates the knot so that it avoids being abraded or broken by adjacent bone structures or appurtenances.

I have found that I can obviate the problems noted in the immediate above paragraph by the following method:

1) Interlocking the loop of the suture with double strands through the tendon which spreads the force of the repair over a larger area of the tendon, in comparison to heretofore methods, so as to improve the pull-out resistance of the suture from the tendon and hence, avoiding the breaking of the suture.

2) Interlocking the loop of the suture and judiciously tying the suture at a prescribed location so as to obtain intimate contact between the tendon and the bone in the bone trough. The method of this invention allows the surgeon to see how closely the tissue is drawn down into the bone trough because the suture is at the end of the tendon instead of underneath the knot. Further, the inventive method allows the surgeon to maintain constant apposition of the tendon to the bone while the knot is being tied or in the case where a clamp is substituted for the knot, while the clamp is advanced toward the securing position.

3) Locating the knot or clamp in the bone trough instead of on top of the tendon where it can be exposed to abrasion from overlying bone or other appurtenances.

### SUMMARY OF THE INVENTION

An object of this invention is to provide an improved method for attaching tissue to the bone to enhance pull-out resistance of the suture to the tendon. The interlocked loop spreads the repair over a larger area of the tendon which serves to improve the pull-out resistance.

A feature of this invention is to interlock the loop of the suture through the tissue and then passing both the suture strands through the anchor eyelet and either knotting the suture downstream of the eyelet or utilizing a clamp in place of the knot.

Another feature of this invention is utilizing one of the strands of the suture to hold the tissue against the bone which strands act as a “post” while tying a half-hitch knot around that “post” to secure the suture, or otherwise tying the other strand to a clamping device.

Another feature of this invention is to improve the healing process of the repair of torn tissue when surgically performed by arthroscopic surgery, by improving the intimate contact between the tendon and the bone trough by tying the suture in a double looped interlocked fashion and knotting or clamping the suture downstream of the anchor which allows the surgeon the visibility and control and feel to cinch the tissue down to the bone and being able to judge or assess how tightly the tissue is apposed to the bone and alleviating the problem of tying the knot and trying to maintain secure apposition of the tendon to the bone.

Another feature of this invention is to employ a double loop in the securing method where both strands of the suture is tied to the tissue and the two strands are inserted into the eyelet of the anchor which is supported in the bone and knotting or clamping the double strand after passing through the eyelet.

The foregoing and other features of the present invention will become more apparent from the following description and accompanying drawings.

### BRIEF DESCRIPTION OF THE DRAWINGS

FIG. 1 is a schematic illustration of the prior art surgical method for tissue repair by attaching tissue to bone utilizing sutures and bone anchors;

FIG. 2 is a fragmentary schematic view illustrating the surgical method of this invention for tissue repair for attaching the tissue to the bone where the knot of the suture is tied downstream of the anchor eyelet;

FIG. 3 is a fragmentary schematic view illustrating of the double strand of the suture sewn through the tissue and the loop formation for cinching the tendon to the bone;

FIG. 4 is a fragmentary schematic view illustrating the subsequent step of the method employed with connection to FIG. 3 showing the pair of strands of a suture passing through the eyelet and secured downstream thereof by a well known surgical knot;

FIG. 5 is a fragmentary schematic view illustrating the identical method where a clamp is substituted for the knot; and

FIG. 6 is an enlarged and perspective view of the clamp shown in FIG. 5.

These figures merely serve to further clarify and illustrate the present invention and are not intended to limit the scope thereof

### DETAILED DESCRIPTION OF THE INVENTION

While the invention describes a method for securing tissue to bone in a surgical procedure and the results are disclosed in the Figs. depicting a particular rivet, it is to be understood that other types of rivets, screws, bolts and securing devices can be employed without impairing the scope of this invention and likewise, the particular knot that is shown in the embodiments are exemplary of any type of suitable knot and this also should not impair the scope of this invention. Suffice it to say that the securing anchor, be it a rivet, pin, screw or the like is embedded in the bone and includes an eyelet for the suture. While this invention is particularly adapted for use in arthroscopic surgery the invention is also applicable in general surgery and the particular suture arrangement can be utilized to not only secure the tissue to bone, but also to tie tissue to other members of the body, such as other tendons or ligaments.

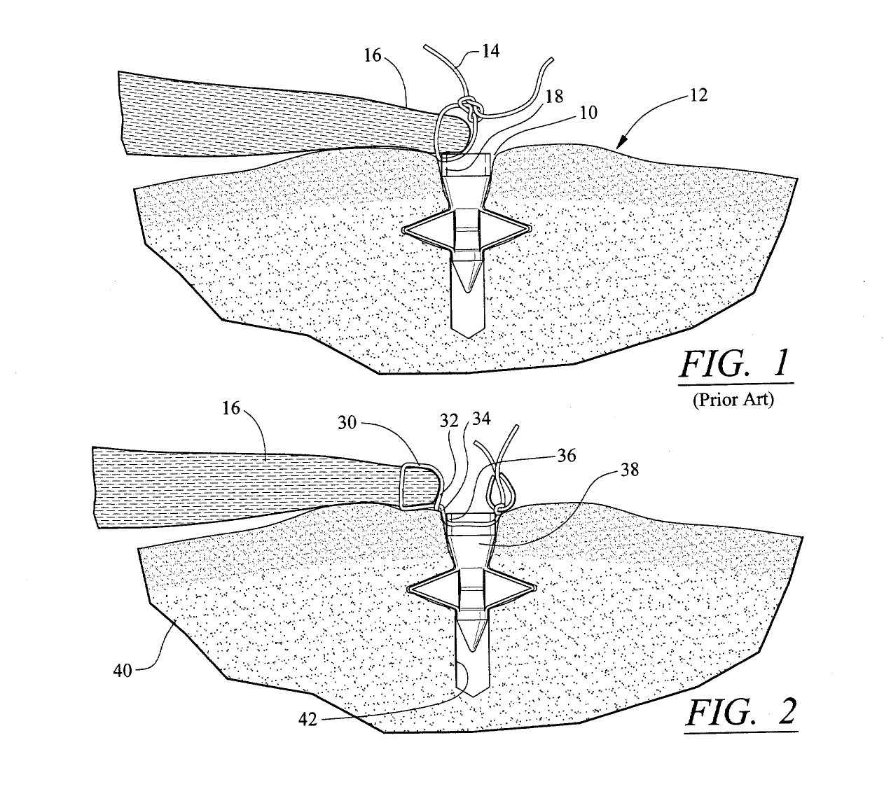

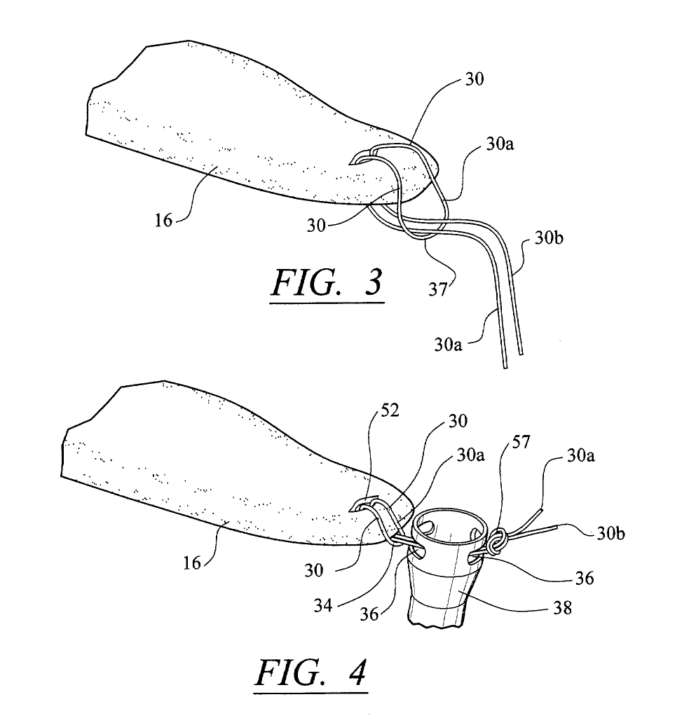

Referring next to FIG. 2 which best shows the method of this invention where the double strands of the suture 30 is sewn into the tendon or tissue 16 by a double loop configuration and drawn through the eyelet 36 of anchor 38 where it is tied with a suitable surgical knot. As is apparent from this FIG. 2, the tendon is cinched to the bone 40 in the pre-prepared trough 42 which has been burred to form a roughen surface in that portion of the bone where growth is promoted. Next, referring to FIGS. 3 and 4 where the view is enlarged and oriented to show the details of the suture 30, it will be appreciated that strands 30a and 30b are interlooped to define a double looped configuration by the following method. The strand 30a is sewn into tendon 16 from the underside and will end up looped under strand 30a and then sewn into tendon 16 from the topside where it now is identified as strand 30b. It is apparent from the foregoing the strands 30a and 30b are drawn through the loop 37. When strands 30a and 30b are drawn together they contact the tendon 16 at point 34 which is tantamount to a pinch point between the tendon and the strands 30a and 30b. This define a “post”. The “post” serves to draw the tendon down to the bone and cinch the tendon to the bone in trough 42. This arrangement of the suture 30 serves to spread the force of the repair over a larger area of the tendon with a consequential improvement in pull-out resistance of the suture from the tendon and also draws the tendon down into the trough 42 in addition to cinching the tendon to the bone. When the repair is done by arthroscopic surgery, one will appreciate that when the suture is being tied and the surgeon is attempting to cinch the tissue down to the bone is difficult for the surgeon to assess how tightly the tissue is apposed to the bone and it is especially difficult to tie the knot while maintaining secure apposition of the tendon to the bone. By virtue of the present invention, this problem is, if not obviated, it is an improvement over the heretofore methods. In this method the surgeon draws the strands 30a and 30b, as shown in FIG. 4, through the both of the eyelets 36 of the rivet anchor 38 and are tied at the downstream end by a suitable surgical knot.

Obviously, the method of obtaining the double loop can be reversed from the direction described or, alternatively, both ends of the suture can be inserted or sewn into the tendon and both stands 30a and 30b are pulled to form the bottom loop, much like a U-shaped member, and the strands are drawn over the tendon and then, into the loop and then pulled to tighten the double loop in the same manner as described in the immediate above paragraph.

It is apparent from the foregoing that since the knot is not over the tendon as is the case shown in FIG. 1 but rather in the trough 40 formed in the bone 40, the likelihood of the knot being abutted by adjacent body parts and hence, broken is significantly reduced, if not eliminated altogether. Of course, if the tissue is not healed before the suture is broken, there is a good likelihood that a surgical repair will be needed and hence, the operation will have to be repeated.

Since the knot is in the trough 42 and away from the tendon and the suture is at the end of the tendon instead of under it, it is readily apparent that the visibility to the surgeon is improved and hence the ability to for the surgeon to draw the tissue and cinch it to the bone trough is greatly improved. Moreover, the ability for the surgeon to maintain a constant apposition of the tendon to the bone while the knot is being tied or the clamp is being installed is significantly improved.

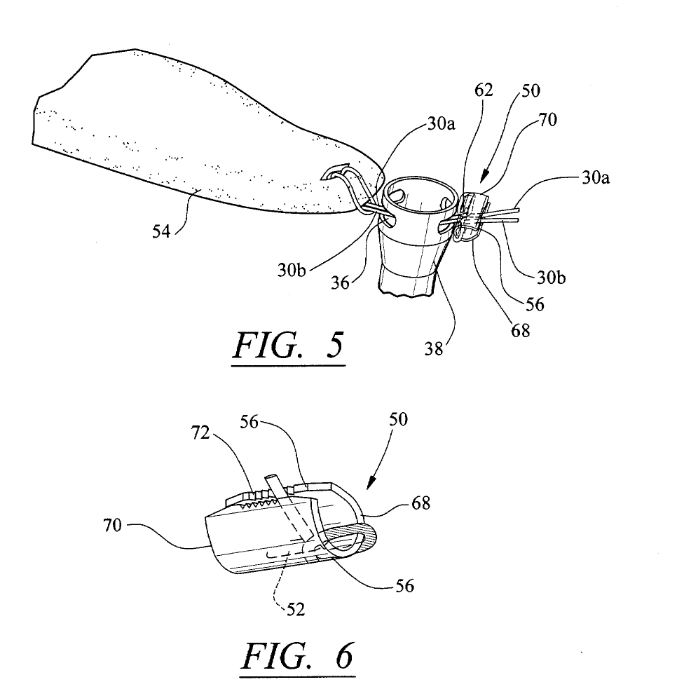

FIG. 5 exemplifies the method where a clamp is used instead of the knot. This clamp 60, or any suitable clamp, can be employed without departing from the scope of the invention. As shown in FIG. 5, strands 30a and 30b pass through both the eyelets 36 of anchor 38. Clamp 50 is then used to secure the suture 30 into place by inserting 30a and 30b into slot 52 formed on the bottom 54 located remote from the slot 66 at the top end thereof and looping strands 30a and 30b around the sides 68 and 70 respectively and then under the bottom 56 and again, through the slot 66 and tightened by pulling strands 30a and 30b from the sides 68 and 70, respectively, and then inserted through the slot 62 and into the serrated portion 72. Both strands 50a and 50b are then drawn through the serrated opening 70. The looping thereof serves to hold the suture in place. Typically, the suture is inserted into the clamp above the wound of the patient and then drawn into the cavity until it rests on the eyelet as shown in Fig.5. A better understanding of this clamp can be had by referring to co-pending patent application entitled “Suture Clip” filed by Eddy Del Rio and myself concurrently herewith and identified as Attorney Docket N1048 and commonly assigned to the same assignee as this patent application and incorporated herein by reference.

It is to be understood herein that the above description relates to the method of tying the tissue to the bone and securing the same as part of an overall operation. The other steps in the operation, as for example, cutting the incision and preparing the cavity and the like are all well known and are not apart of the method, although they are performed as part of the surgical procedure.

Although this invention has been shown and described with respect to detailed embodiments thereof, it will be appreciated and understood by those skilled in the art that various changes in form and detail thereof may be made without departing from the spirit and scope of the claimed invention.

It is claimed:

1. The method of tying torn tissue with suture to a bone that has been prepared by burring a trough in the bone and fixing an anchor in or in close probity to the trough and tying the suture to the anchor comprising the steps of:
i) sewing the tendon with suture so that the suture forms a double loop over the tendon and is drawn through a loop defined by the suture;
ii) drawing the suture through eyelet of the anchor so that the juncture of the suture and the double loop is adjacent the bone and the ends of the suture is downstream of the anchor; and
iii) tying the ends of the suture together with a surgical knot.

2. The method as claimed in claim 1 including a clamp replacing the knot claimed in the step iii.

3. The method of tying torn tissue with suture to a bone that has been prepared by burring a trough in the bone and fixing an anchor in or in close proximity to the trough and tying the suture to the anchor comprising the steps of:
i) cutting through the skin and tissue of the patient to expose the area of the patient including the adjacent bone that requires repair of a tendon or ligament;
ii) burring a surface of the bone exposed in step i to form a trough and a roughened surface of the bone;
iii) inserting an anchor into the trough formed in step ii.
iv) sewing the tendon or ligament with a suture so that two portions of the suture pass through the tendon or ligament and form a loop and drawing the two portions of the suture through the loop;
v) drawing the two portions of the suture extending downstream of the loop into an eyelet of the anchor; and
vi) tying the two portions of the suture downstream of the eyelet to form a knot to secure the tendon or ligament to cinch to the bone of the patient.

4. The method of tying torn tissue with suture to a bone that has been prepared by burring a trough in the bone and fixing an anchor in or in close proximity to the trough and tying the suture to the anchor comprising the steps of:
i) cutting through the skin and tissue of the patient to expose the area of the patient including the adjacent bone that requires repair of a tendon or ligament;
ii) burring a surface of the bone exposed in step i to form a trough and a roughened surface of the bone;
iii) inserting an anchor into the trough formed in step ii.
iv) sewing the tendon or ligament with a suture so that two portions of the suture pass through the tendon or ligament and form a loop and drawing the two portions of the suture through the loop;
v) drawing the two portions of the suture extending downstream of the loop into an eyelet of the anchor; and
vi) tying the two portions of the suture downstream of the eyelet to form a knot to secure the tendon or ligament to cinch to the bone of the patient.
